(19) United States
(12) Patent Application Publication
(10) Pub. No.: US 2009/0163742 A1
(43) Pub. Date: Jun. 25, 2009

(54) HETEROGENEOUS PROMOTION OF OXIRANE HYDROFORMYLATION

(76) Inventors: Stephen Blake Mullin, Magnolia, TX (US); Joseph Broun Powell, Houston, TX (US); Paul Richard Weider, Houston, TX (US)

Correspondence Address: SHELL OIL COMPANY, P O BOX 2463, HOUSTON, TX, 772522463, US

(21) Appl. No.: 12/337,374

(22) Filed: Dec. 17, 2008

Related U.S. Application Data
(60) Provisional application No. 61/014,766, filed on Dec. 19, 2007.

(51) Int. Cl. C07C 45/00

(52) U.S. Cl. 568/485

(57) ABSTRACT

A process for making betahydroxyaldehydes such as 3-hydroxypropanal which comprises intimately contacting (a) an oxirane, (b) carbon monoxide, (c) a reducing agent such as hydrogen, (d) from about 0.01 to about 1 weight percent, basis cobalt metal, of a cobalt hydroformylation catalyst which is optionally complexed with a tertiary phosphine ligand, and (e) a heterogeneous, preferably solid, metal promoter used at a molar ratio of 0.05, preferably 0.15, to 100 moles of heterogeneous metal relative to the moles of soluble cobalt hydroformylation catalyst.

This application claims the benefit of U.S. Provisional Application No. 61/014,766, filed Dec. 19, 2007, which is incorporated herein by reference.

### FIELD OF THE INVENTION

This invention relates to a process for making 3-hydroxypropanal (or other betahydroxyaldehydes) and, ultimately, 1,3-propanediol (or other 1,3-alkane diols) by hydroformylating oxiranes such as ethylene oxide using a cobalt carbonyl catalyst.

### BACKGROUND OF THE INVENTION

3-hydroxypropanal and other betahydroxyaldehydes are useful chemical intermediates. The former can be readily converted to 1,3-propane diol which finds use as an intermediate in the production of polytrimethylene terephthalate which is used to make fibers, textiles and carpets.

U.S. Pat. Nos. 3,456,017, 3,463,819, and 5,256,827 teach processes for the hydroformylation of ethylene oxide to produce 3-hydroxypropanal or betahydroxyaldehydes and, ultimately, 1,3-propane diol or other 1,3-glycols using tertiary phosphine-modified cobalt carbonyl catalysts. One of the disadvantages of these processes is that the catalysts also promote the undesired re-arrangement of ethylene oxide to acetaldehyde which is an undesirable byproduct. At conventional operating pressures such as 10 MPa or less the reaction is only about 80 to 85% selective to 3-hydroxypropanal. As much as 15-18 molar percent of this byproduct may be produced.

U.S. Pat. No. 5,256,827 in particular describes the use of C₂-bridged bidentate phosphine ligands, such as 9-phosphabicyclo[3.3.1]nonane to increase the activity of the cobalt catalyst and to produce 3-hydroxypropanal in high molar yields such as 70 to 99 percent. One of the advantages of using these particular bidentate phosphine ligands is that they suppress the undesired re-arrangement of ethylene oxide to acetaldehyde under the conditions of hydroformylation. These ligands are expensive and subject to thermal degradation or losses in catalyst recovery and recycle.

Thus, there is a need for a method of hydroformylation of oxiranes such as ethylene oxide to 3-hydroxypropanal or other betahydroxyaldehydes which accelerates rates and allows use of smaller reactors at lower capital cost. Increasing the rate of hydroformylation can also increase the yield of the desired product relative to undesired re-arrangement of ethylene oxide to acetaldehyde.

### SUMMARY OF THE INVENTION

This invention provides a process for making betahydroxyaldehydes such as 3-hydroxypropanal which comprises intimately contacting, preferably in liquid phase solution in an inert reaction solvent,

(a) an oxirane, preferably with 2 to 10 carbon atoms, most preferably ethylene oxide,

(b) carbon monoxide,

(c) a reducing agent such as hydrogen,

(d) from about 0.01 to about 1 weight percent, basis cobalt metal, preferably about 0.1 to about 0.5 weight percent, of a cobalt hydroformylation catalyst which is optionally complexed with a tertiary phosphine ligand, and

(e) a heterogeneous, preferably solid, metal promoter used at a molar ratio of about 0.05, preferably about 0.15, to about 100 moles of heterogeneous metal relative to the moles of soluble cobalt hydroformylation catalyst. The heterogeneous solid promoter may be selected from the group consisting of Raney or sponge metal cobalt, Raney copper, Raney nickel, supported cobalt, supported copper, supported nickel, supported ruthenium, supported iron, and supported rhodium catalysts. The temperature may range from about 30 to about 100° C. and the pressure may range from about 1.5 to about 25 MPa.

While much of the description herein refers to ethylene oxide, 3-hydroxypropanal and 1,3-propanediol, this invention is also applicable to oxiranes, betahydroxyaldehydes, and 1,3-glycols, respectively, in general.

### DETAILED DESCRIPTION OF THE INVENTION

An oxirane such as ethylene oxide is hydroformylated by reaction with carbon monoxide and a reducing agent such as hydrogen in the presence of a homogeneous (soluble) cobalt carbonyl catalyst which is optionally modified with a tertiary phosphine ligand. The reaction products comprise primarily 3-hydroxypropanal or other betahydroxyaldehyde and, ultimately, 1,3-propanediol or other 1,3-glycol. The ratio of the two products can be adjusted by adjusting the amount of catalyst present in the reaction mixture, the reaction temperature or the amount of hydrogen pressure in the reaction. When the term “3-hydroxypropanal” is used herein it is understood to mean this monomer as well as dimers thereof, such as 2-(2-hydroxyethyl)-4-hydroxy-1,3-dioxane, as well as trimers and higher oligomers of 3-hydroxypropanal. In a preferred embodiment, lower amounts of catalyst are used to produce primarily the aldehyde and its oligomers which are then hydrogenated to the 1,3-propanediol in a separate hydrogenation step using a conventional hydrogenation catalyst and hydrogen. The optional use of tertiary phosphine ligands as complexing ligands for the cobalt catalyst results in catalysts with increased activity and this results in more ethylene oxide being converted to product aldehyde and diol.

The homogeneous (soluble) cobalt hydroformylation catalyst may be prepared by a diversity of methods. These catalysts and methods for making them are described in U.S. Pat. No. 5,256,827, which is herein incorporated by reference in its entirety.

A convenient method is to combine a cobalt salt, organic or inorganic, with the phosphine ligand, if it is used, in liquid phase followed by reduction and carbonylation. Suitable cobalt salts comprise for example, cobalt carboxylates such as acetates, octanoates, etc. which are preferred, as well as cobalt salts of mineral acids such as chlorides, fluorides, sulphates, sulphonates, etc. Operable also are mixtures of these cobalt salts. It is preferred, however, that when mixtures are used, at least one component of the mixture be a cobalt alkanoate of 6 to 12 carbon atoms. The valent state of the cobalt may be reduced and the cobalt-containing complex formed by heating the solution in an atmosphere of hydrogen and carbon monoxide. The reduction may be performed prior to the use of the catalyst or it may be accomplished simultaneously with the hydroformylation process in the hydroformylation zone.

Alternatively, the catalyst may be prepared from a carbon monoxide complex of cobalt if a suitable phosphine ligand is to be used. For example, it is possible to start with dicobalt octacarbonyl and, by heating this substance with a suitable phosphine ligand, the ligand replaces one or more, preferably at least 2, of the carbon monoxide molecules, producing the desired catalyst. When this latter method is executed in a hydrocarbon solvent, the complex may be precipitated in crystalline form by cooling the hot hydrocarbon solution. This method is very convenient for regulating the number of carbon monoxide molecules and phosphine ligand molecules in the catalyst. Thus, by increasing the proportion of phosphine ligand added to the dicobalt octacarbonyl, more of the carbon monoxide molecules are replaced.

The heterogeneous (solid) metal promoter may be produced by any suitable means used to prepare a heterogeneous catalyst of high surface area, including impregnation or precipitation on a high surface area support, coprecipitation of metal with support, and leaching of metal alloys (metal-aluminum) as practiced in the preparation of Raney™ type or sponge metal catalysts. Sponge metal is a finely divided and porous form of metal made by decomposition or reduction without melting.

The heterogeneous, preferably solid, promoter material may be selected from the group consisting of Raney or sponge metal cobalt, Raney copper, Raney nickel, supported cobalt, supported copper, supported nickel, supported ruthenium, supported iron, and supported rhodium catalysts. The preferred catalysts are Raney or sponge metal cobalt and supported cobalt, copper and ruthenium catalysts. Most preferably, the heterogeneous cobalt promoter is comprised of Raney or sponge metal cobalt.

The heterogeneous hydroformylation promoter may be present in a molar ratio of from about 0.01 to about 100 moles of heterogeneous metal relative to the moles of soluble hydroformylation catalyst present. Preferably, the molar ratio is from about 0.15 to about 100.

The heterogeneous promoter may be contacted with the soluble homogeneous hydroformylation mixture via any configuration practiced for effecting gas-liquid-solid contacting in chemical reacting systems. If deployed as a supported promoter or “catalyst” in a fixed bed configuration, the molar amount of solid metal promoter in the reactor relative to the concentration of soluble cobalt hydroformylation catalyst may range from about 0.1 to 100. The heterogeneous promoter may for example be deployed as a trickle bed, with thin films of liquid containing the soluble cobalt hydroformylation catalyst flowing downward over the fixed bed of solid promoter. The highest ratio of solid promoter to soluble hydroformylation catalyst is obtained in this configuration. Alternately, liquid containing soluble hydroformylation catalyst may flow upwards over a fixed or fluidized bed of solid promoter together with synthesis gas (carbon monoxide and hydrogen) required to effect hydroformylation. If the flow of liquid solution plus gas does not exceed the minimum velocity for fluidization of the bed, then this is known as a fixed-bed bubble column configuration. For either fixed bed bubble column or trickle bed operating regimes, the heterogeneous promoter may comprise greater than 65% of the reactor volume for typical supported catalysts. If the particle size of the heterogeneous promoter is reduced or the flowrate of the gas or liquid increased, the bed of heterogeneous promoter will expand and fluidize to obtain a “fluidized bed” configuration, where the heterogeneous promoter will typically comprise 10 to 70% of the reactor volume. If the particle size is further reduced to obtain finely divided particles of typical 1 to 100 micron size, the operating regime of a “slurry reactor” is obtained, where the concentration of heterogeneous promoter in the reactor comprises typically 1 to 10% of the total reactor volume. Use of finely divided sponge metal or Raney-type catalysts comprise operation in the slurry-reactor regime. In addition to use of liquid and gas velocity to mix and suspend catalyst, a mechanical stirrer or jet-loop mixer may be used to suspend the slurry of heterogeneous promoter to assure intimate contacting with the hydroformylation mixture.

Other possible support configurations include the use of catalytic support monoliths or coated structured packings, where the volume fraction of fixed heterogeneous promoter is much lower than that obtained with a bed of particles. Monolithic or structured supports may comprise only 5 to 50% of the reactor volume and have the advantage of allowing more volume for the hydroformylation mixture with soluble hydroformylation catalyst.

In an alternate embodiment, the soluble hydroformylation catalyst may be passed over a fixed or fluidized bed of heterogeneous promoter, with the fixed bed promoter comprising more than 65% of the cross section of the vessel.

Support materials which can be used for the promoter of this invention include oxide supports and activated carbon supports. Examples of oxide materials which are suitable as the support material for the promoter include titanium dioxide, silicon dioxide, aluminum trioxide and/or mixed oxides comprising at least two members selected from this group, for example, aluminum silicate. Other suitable oxide materials include silica gel, magnesium oxide, zeolites and/or zirconium dioxide. Activated carbon supports suitable for the preparation of the carbon-supported metal catalysts are described in U.S. Pat. No. 6,297,408 which is herein incorporated by reference in its entirety. Activated carbons are in general made from carbonized biopolymers which are activated, for example, by steam activation or chemical activation, to generate micropores of various size and shape distribution. The pore volume of the activated carbons depends on the starting material and the activation process used. Preferred support materials include silica, alumina, silica-alumina, titania, zirconia and activated carbon. Fixed monolithic supports or structured packings may also be use as support for the heterogeneous solid promoter.

From about 0.01 to about 1 weight percent, basis cobalt metal, generally from about 0.1 to about 0.5 weight percent, of the homogeneous cobalt carbonyl catalyst may be used, with the heterogeneous promoter used at a ratio of about 0.05 to about 100, the basis being moles of heterogeneous metal relative to the moles of soluble cobalt hydroformylation catalyst. In order to achieve the advantages of this invention in terms of promotion of the ethylene oxide hydroformylation and minimization of the isomerization of ethylene oxide to acetaldehyde, the molar ratio of the metal from the heterogeneous promoter to the soluble cobalt from the cobalt carbonyl hydroformylation catalyst should be greater than 0.05, preferably greater than about 0.15. If the weight percent of heterogeneous promoter is less than about 0.05% the ethylene oxide hydroformylation rate will not be increased significantly.

Other soluble promoters such as amines, ammonium and phosphonium salts may also be present in ratio of about 0.05 to 0.5 moles per mole of soluble hydroformylation catalyst. One example is dimethyldodecylamine.

Infrared spectra show that at steady state, peaks associated with dicobaltoctacarbonyl (@ 2072 cm-1) virtually vanish from the spectrum with use of the solid promoters described in this invention. This contrasts the continuing presence of peaks associated with dicobaltoctacarbonyl when soluble amine, ammonium, or phosphonium promoters are used. Thus, it appears that a fundamental shift in reaction mechanism has occurred. One also notes a fairly significant increase in overall reaction rate suggesting that the rate determining step has been altered. This has led us to theorize that the heterogeneously promoted hydroformylation reaction proceeds according an altered mechanism or via a change in rate limiting step.

If it is desired to use a tertiary phosphine stabilizing ligand with the cobalt carbonyl catalyst, the ligand may be chosen from those described in U.S. Pat. No. 5,256,827 which is herein incorporated by reference in its entirety. In one embodiment, the stabilizing ligand is a tertiary phosphine of a single phosphorus atom as the sole complexing site in the tertiary phosphine ligand. This class of tertiary phosphines, herein termed monophosphines, is generically classified as tertiary monophosphines of from 3 to 60 carbon atoms wherein each phosphorous substituent is a hydrocarbon substituent, i.e., contains only atoms of carbon and hydrogen. A preferred class of tertiary monophosphines is represented by the formula:

RRRP (I)

wherein R independently is monovalent hydrocarbon (i.e., “hydrocarbyl”) of up to 30 carbon atoms, preferably up to 20, more preferably up to 12, with the proviso that two R may together form a divalent hydrocarbon moiety of up to 60 carbon atoms. Such cyclophosphines are illustrated by 1-ethylphospholidine, 1-phenylphospholidine, 1-phenylphosphorinane, 1-butylphosphorinane, 4,4-dimethyl-1-phenylphosphorinane, 1-phenyl-phosphepane, 1-ethylphosphepane, 3,6-dimethyl-1-phenylphosphepane, 9-phenyl-9-phosphabicyclo[3.3.1]nonane and 9-butyl-9-phosphabicylco[4.2.1]nonane.

In another embodiment of the phosphine-modified cobalt complexes which may be used in this invention, the tertiary phosphine employed is a bidentate ligand, i.e., the phosphine ligand is a tertiary diphosphine. A suitable broad class of tertiary diphosphine ligands are described in great detail in U.S. Pat. No. 5,256,827 which is herein incorporated by reference in its entirety. Tertiary phosphine ligands may also be employed as a polydentate ligand. Such tertiary phosphine ligands are also described in great detail in the aforementioned U.S. Pat. No. 5,256,827.

As described in U.S. Pat. No. 5,256,827, a particularly preferred ditertiary phosphine complexing ligand comprises a hydrocarbylene-bis(monophosphabicycloalkane) in which each phosphorus atom is joined to hydrocarbylene and is a member of a bridge linkage without being a bridge head atom and which hydrocarbylene-bis(monophosphabicycloalkane) has 11 to 300. 5 to 12 carbon atoms thereof together with a phosphorus atom are members of each of the two bicyclic skeletal structures. Particularly preferred ditertiary phosphines are 9-phosphabicyclo[4.2.1]nonane and 9-phosphabicyclo[3.3.1]nonane. The term “hydrocarbylene” is used herein in its accepted meaning as representing a diradical formed by removal of 2 hydrogen atoms from a carbon atom or preferably 1 hydrogen atom from each of two different carbons of a hydrocarbon.

According to U.S. Pat. No. 5,256,827, it is preferred to partially oxidize the tertiary phosphine ligands to convert the phosphines in part to phosphine oxide. The oxidation is carried out with an oxidant under mild oxidizing conditions such that an oxygen will bond to a phosphorus, but phosphorus-carbon, carbon-carbon and carbon-hydrogen bonds will not be disrupted. By suitable selection of temperatures, oxidants and oxidant concentrations such mild oxidation can occur. The oxidation of phosphine ligands may be carried out prior to the forming of the catalyst complex. Suitable oxidizing agents include peroxy-compounds, persulfates, permanganates, perchromates, and gaseous oxygen.

The optimum ratio of ethylene oxide feed to cobalt carbonyl catalyst will in part depend upon the particular cobalt carbonyl catalyst and operating conditions of temperature and pressure employed. However, molar ratios of ethylene oxide to cobalt carbonyl catalyst from about 2:1 to 10,000:1 are generally satisfactory, with molar ratios of from about 50:1 to about 500:1 being preferred. When batch processes are used, it is understood that the above ratios refer to the initial starting conditions. In one modification, a cobalt carbonyl complex is employed as a preformed material, being prepared by reaction of a cobalt salt with carbon monoxide and hydrogen in the presence of a phosphine ligand and then isolated and subsequently utilized in the present process. In another embodiment utilizing a ligand, the phosphine-modified cobalt complex is prepared in situ as by addition to the reaction mixture of a cobalt salt or a cobalt octacarbonyl together with the phosphine ligand whose introduction into the catalyst complex is desired.

If a phosphine ligand is to be used, it is preferable to employ the phosphine-modified cobalt complex in conjunction with a minor proportion of excess tertiary phosphine ligand (oxidized or unoxidized) which is the same as or is different from the phosphine ligand of the cobalt complex. Although the role of the excess phosphine is not known with certainty, the presence thereof in the reaction system appears to promote or otherwise modify catalyst activity.

Phosphorus:cobalt (from the homogeneous cobalt catalyst) atom ratios used in conjunction with the catalyst complex will range from about 1:1 to about 3:1, preferably from about 1.2:1 to about 2.5:1. A ratio of about 2:1 is particularly preferred.

The heterogeneous metal promoter may be retained in the reaction mixture as a slurry catalyst, with separation and recycle via filtration or gravity separation, or used as a fluidized bed or fixed-bed catalyst.

The process of the invention may be conducted in liquid phase solution in an inert solvent. Although a variety of solvents which are inert to the reactants and catalyst and promoter and which are liquid at reaction temperature and pressure are in part operable, illustrative of suitable solvents are hydrocarbons, particularly aromatic hydrocarbons of up to 16 carbon atoms such as benzene, toluene, xylene, ethylbenzene, and butylbenzenes; alkanes such as hexanes, octanes, dodecanes, etc.; alkenes such as hexenes, octenes, dodecenes, etc.; alcohols such as tertiary butyl alcohol, hexanol, dodecanol, including alkoxylated alcohols; nitrites such as acetonitrile, propionitrile, etc.; ketones, particularly wholly aliphatic ketones, i.e., alkanones, of up to about 16 carbon atoms such as acetone, methylethylketone, diethylketone, methylisobutylketone, ethyhexylketone and dibutylketone; esters of up to 16 carbon atoms, particularly lower alkyl esters of carboxylic acids which are aliphatic or aromatic carboxylic acids having one or more carboxyl groups, preferably from 1 to 2, such as ethylacetate, methylpropionate, propylbutyrate, methylbenzoate, diethylglutarate, diethylphthalate, and dimethylterephthalate; and ethers of up to about 16 carbon atoms and up to about 4 ether oxygen atoms, which ethers are cyclic or acyclic ethers and which are wholly aliphatic ethers, e.g., diethylether, diisopropylether, dibutylether, ethylhexylether, methyloctylether, dimethoxyethane, diethyleneglycoldimethylether, diethylenediethylether, diethyleneglycoldibutylether, tetraglyme, glyceroltrimethylether, 1,2,6-trimethoxyhexane, tetrahydrofuran, 1,4-dioxane, 1,3-dioxane, 1,3-dioxylene and 2,4-dimethyl-1,3-dioxane, or which are at least partially aromatic, e.g., diphenylether, phenylmethylether, 1-methylnapthalene, phenylisopropylether, halogenated hydrocarbons, such as chlorobenzene, dichlorobenzene, fluorobenzene, methylchloride, and methylenechloride. Mixtures of solvents can also be utilized. The amount of solvent to be employed is not critical. Typical molar ratios of reaction solvent to ethylene oxide reactant may vary from about 5:1 to about 150:1.

Suitable selection of solvents may enhance product recovery. By selecting solvents with suitable polarity, a two-phase system will form upon cooling of the reaction mixture with selective distribution of the catalyst and ligand, if present, in one phase and product 3-hydroxypropanal and 1,3-propane diol in a second phase. This will allow for easier separation of catalysts and recycle thereof back to the first stage reactor. If thermal separation (distillation) is used to separate hydroformylation catalyst from product, the solid promoter may be implemented in slurry form and recycled with phosphine-ligated catalyst as the heavy bottoms from distillation. Most preferably, the solid promoter is separated prior to product separation via use of filtration or gravity separation means. The solid promoter may be implemented as a fluidized bed so that no separation is required. A fixed bed may also be employed. When a two-phase liquid-liquid separation process is used, solvents that would not be desirable in the reaction mixture, such as water and acids, can be used to enhance distribution of product to one-phase and catalyst to the other phase. Illustrative solvents for use in a one-phase system are diethylene glycol, tetraglyme, tetrahydrofuran, tertiary butyl alcohol and dodecanol. Illustrative solvents for use to provide a two-phase system upon cooling are toluene, 1-methylnaphthalene, xylenes, diphenylether, and chlorobenzene.

The process of the invention comprises contacting the ethylene oxide reactant, soluble catalyst and heterogeneous catalyst promoter and with carbon monoxide and molecular hydrogen. The molar ratio carbon monoxide to hydrogen most suitably employed is from about 4:1 to about 1:6 with best results being obtained when ratios of from about 1:1 to about 1:4 are utilized. No special precautions need to be taken with regard to the carbon monoxide and hydrogen and commercial grades of these reactants are satisfactory. The carbon monoxide and hydrogen are suitably employed as separate materials although it is frequently advantageous to employ commercial mixtures of these materials, e.g., synthesis gas.

The addition of small amounts of acids and alkali metal salts to the hydroformylation reaction mixture may further enhance or promote the conversion of ethylene oxide by increasing the activity of the catalyst. Acids are defined herein to mean those compounds which may donate a proton under reaction conditions. In general, any alkali metal salt that does not react with ethylene oxide, the reaction solvent, or the hydroformylation products may be suitable as a copromoter with the acids. These are described in U.S. Pat. No. 5,256,827 which is herein incorporated by reference.

In a preferred embodiment, the product of the hydroformylation reaction is further hydrogenated to produce a product comprising substantially 1,3-propane diol. The hydroformylated product is preferably separated from the catalyst before being hydrogenated. Inert solvent may be added to the product prior to hydrogenation, or, if an inert (to hydrogenation) solvent was used in the hydroformylation reaction, it may be separated with the product and passed to the hydrogenation reactor. The hydrogenation catalyst may be any of the well known hydrogenation catalyst used in the art such as Raney nickel, palladium, platinum, ruthenium, rhodium, cobalt and the like. Preferred catalysts are Raney nickel and supportive platinum, particularly platinum on carbon. These are described in U.S. Pat. No. 5,256,827 which is herein incorporated by reference.

One of the considerations of the present invention is that the reaction may be carried out under relatively mild hydrogenation conditions for initial conversion, followed by a high temperature condition where byproducts are reverted to diol product. The process may be carried with an initial temperature of from about 30 to about 100° C., preferably from about 35 to about 80° C., and then optionally at a final temperature in the range of about 120 to about 175° C. The temperature may be increased to revert heavy byproducts to diol product when a majority of the betahydroxyaldehye has been converted to the corresponding diol. The reaction may be carried out at a pressure of at least 0.8 MPa, generally within the range of about 1.5 to 25 MPa.

### EXAMPLES

### Examples 1 to 18

A series of hydroformylation experiments were conducted in 100- or 300-ml stirred reactors with hollow shaft draft-tube gas dispersion. The reactions were conducted at 65-80° C., with 10 MPa of 4:1 molar H2/CO synthesis gas. The reactors were charged under inert atmosphere with 40-50% by volume liquid MTBE solvent containing 0.1-0.2 wt % cobalt as dicobaltoctacarbonyl, optional dimethydrodecylamine promoter at promoter (Pr)/cobalt molar ratio (Pr/Co) of 0.2-0.4, optional deionized water (zero or 1 weight percent), and one or more solid promoters in the experiments according to this invention. Ethylene oxide was dosed directly via calibrated sight glass (300-ml) or as a diluted mixture in MTBE solvent via a 6-port sample injection valve with a 2.5-ml sample loop (100-ml reactors). For those studies conducted at temperatures other than 70° C., the hydroformylation reaction rates were adjusted to this temperature assuming an activation energy of 34 kcal/gmol as determined in separate experiments. The reactions were continued until the synthesis gas consumption rate slowed, indicating conversion of more than 90% of the ethylene oxide charge. Gas Chromatography (GC) analysis was used to assess the formation of hydroformylation products 3-hydroxypropanal (HPA) and 1,3-propanediol (PDO), and byproducts acetaldehyde and ethanol. The reaction rates were assessed as a turnover frequency (TOF), expressed as moles of hydroformylation products (HPA and PDO) per mole of cobalt per hour.

The results are shown in Table 1. The reaction rates (TOF) at 70° C. ranged from 1-4/h (per hour) for the dry, unpromoted system (comparative examples 1 to 3; example 3 at 0.06 Pr/Co ratio was too little soluble promoter to be effective). Addition of dimethydrodecylamine soluble promoter at concentrations above 0.2 Pr/Co gave TOR on the order of 5 to 7/h under dry conditions (comparative examples 4 and 5), and 8 to 11/h under wet conditions with 1% by weight water added (comparative examples 6 to 10).

Solid Raney™ cobalt, as well as supported copper/silica and ruthenium/carbon catalysts were examined as solid promoters (Examples 11-18). Turnover frequencies (70° C.) ranged from 8 to 16/h, which is a 2- to 4-fold increase in rate relative to the unpromoted examples 1 to 3. Raney cobalt exhibited the highest promotional effect. Yields of hydroformylation products were in many cases enhanced via use of solid promotion. Post reaction analyses indicated no measurable increase in soluble cobalt where solid cobalt catalysts were employed.

TABLE 1
Large Batch Tests of Heterogeneous at 70° C.

| | | | Solid | | Solid | | | |
|---|---|---|---|---|---|---|---|---|
| | | Solid | #2 | Soluble | Promoter | | Yield | Rate |
| | Solid | promoter | Cu—Si | molar | molar | H2O | % HF | (TOF) |
| Ex # | promoter | wt % | wt % | Pr/Co | Pr/Co | wt % | Products | 1/h |
| 1 | none | 0.00% | 0.00% | 0.00 | 0.00% | 0.00% | 76% | 4.4 |
| 2 | none | 0.00% | 0.00% | 0.00 | 0.00% | 0.00% | 56% | 1.3 |
| 3 | none | 0.00% | 0.00% | 0.06 | 0.00% | 0.00% | 80% | 3.0 |
| 4 | none | 0.00% | 0.00% | 0.22 | 0.00% | 0.00% | 85% | 6.4 |
| 5 | none | 0.00% | 0.00% | 0.27 | 0.00% | 0.00% | 81% | 5.6 |
| 6 | none | 0.00% | 0.00% | 0.32 | 0.00% | 1.00% | 76% | 8.5 |
| 7 | none | 0.00% | 0.00% | 0.35 | 0.00% | 1.00% | 77% | 9.8 |
| 8 | none | 0.00% | 0.00% | 0.25 | 0.00% | 1.00% | 79% | 10.4 |
| 9 | none | 0.00% | 0.00% | 0.35 | 0.00% | 1.00% | 77% | 9.8 |
| 10 | RaCo | 8.33% | 0.00% | 0.00 | 37.5 | 0.00% | 78% | 15.1 |
| 11 | RaCo | 8.33% | 0.00% | 0.00 | 37.5 | 0.00% | 70% | 13.9 |
| 12 | RaCo | 4.17% | 0.00% | 0.00 | 14.0 | 0.00% | 76% | 13.6 |
| 13 | CuSi | 0.00% | 3.97% | 0.00 | 1.6 | 0.00% | 90% | 8.3 |
| 14 | 5% RuC | 3.97% | 0.00% | 0.00 | 1.2 | 0.00% | 78% | 9.9 |
| 15 | RaCo | 4.17% | 0.00% | 0.00 | 37.5 | 0.00% | 84% | 9.9 |
| 16 | RaCo | 2.08% | 2.08% | 0.00 | 22.5 | 0.00% | 89% | 8.2 |
| 17 | RaCo | 2.08% | 4.17% | 0.00 | 26.3 | 0.00% | 90% | 9.0 |
| 18 | CuSi | 0.00% | 4.20% | 0.00 | 7.6 | 1.00% | 87% | 6.4 |

RaCo = Raney ™ cobalt slurry catalyst (WR Grace 2724, Cr-promoted)
CuSi = Engelhard Cu-0602 copper/silica catalyst, crushed
5% RuC = Precious Metal Catalysts/Activated Metals Inc. # 3110C
Soluble promoter = dimethyldodecylamine

### Example 19 to 36

### Multi-Throughput Reactor Tests

A similar set of experiments were conducted in a 6-station multireactor of 75-ml volume. For these experiments, 2:1 H2/CO ratio was used for synthesis gas. All reactions were conducted dry, with 0.27 weight percent cobalt as dicobaltoctacarbonyl added as hydroformylation catalyst. The results again showed that solid cobalt or copper catalysts can promote the hydroformylation reaction in absence of soluble amine promoter.

TABLE 2
Multi-Reactor Tests of Solid Promoter

| | | Soluble | Solid | | | |
|---|---|---|---|---|---|---|
| | | Promoter | Promoter | | Yield HF | Rate |
| | | Ratio | Ratio | Temp | Products | (TOF) |
| Ex # | Solid Promoter | Pr/Co | Pr/Co* | (° C.) | (%) | 1/H |
| 19 | None | 0.00 | 0.0 | 80 | 21.0% | 0.33 |
| 20 | 6 wt % Cu/SiO2 | 0.00 | 6.0 | 80 | 73.7% | 5.16 |
| 21 | 6 wt % Cu/SiO2 | 0.30 | 6.0 | 80 | 79.4% | 4.63 |
| 22 | 6 wt % RaCo | 0.00 | 27.6 | 80 | 63.4% | 9.12 |
| 23 | 6 wt % RaCo | 0.30 | 27.6 | 80 | 61.0% | 8.74 |
| 24 | None | 0.30 | 0.0 | 80 | 54.4% | 6.37 |
| 25 | None | 0.00 | 0.0 | 80 | 34.8% | 0.66 |
| 26 | 6 wt % CuSi | 0.00 | 6.0 | 80 | 81.5% | 4.96 |
| 27 | 6 wt % CuSi | 0.30 | 6.0 | 80 | 83.6% | 5.43 |
| 28 | 6 wt % RaCo | 0.00 | 27.6 | 80 | 67.1% | 11.72 |
| 29 | 6% RaCo | 0.30 | 27.6 | 80 | 68.0% | 11.18 |
| 30 | None | 0.30 | 0.0 | 80 | 51.9% | 6.38 |
| 31 | None | 0.00 | 0.0 | 75 | 65.8% | 0.74 |
| 32 | 3 wt % CuSi + | 0.00 | 16.8 | 75 | 84.4% | 8.22 |
| | 3 wt % RaCo | | | | | |
| 33 | 3 wt % CuSi + | 0.30 | 16.8 | 75 | 85.7% | 7.40 |
| | 3% RaCo | | | | | |
| 34 | 6% CuSi + | 0.00 | 33.6 | 75 | 83.8% | 8.22 |
| | 6 wt % RaCo | | | | | |
| 35 | 10 wt % CuSi | 0.30 | 10.0 | 75 | 73.1% | 1.48 |
| 36 | None | 0.30 | 0.0 | 75 | 76.4% | 9.82 |

*Approximate assuming 20 wt % Cu on silica
Soluble promoter = dimethyldodecylamine
Solid catalyst weight percents expressed relative to total mass of liquid charged.

The WR Grace 2724 Cr-promoted Raney™ cobalt catalyst used above has the following composition.

### Aluminum % 3.21

### Cobalt % 92.48

### Chromium % 2.05

### Iron % 0.26

### Nickel % 2.00

27.58 microns

Base metal catalysts effective for use in this invention include nickel-, cobalt-, or copper-aluminum alloy catalysts. A preferred catalyst is a cobalt-aluminum alloy catalyst such as that sold as Raney cobalt catalyst by Engelhard Corporation.

We claim:

1. A process for making a betahydroxyaldehyde, preferably 3-hydroxypropanal, which comprises intimately contacting, preferably in liquid phase solution in an inert reaction solvent,
(a) an oxirane
(b) carbon monoxide,
(c) a reducing agent such as hydrogen
(d) from 0.01 to 1 weight percent, basis cobalt metal, of a cobalt hydroformylation catalyst which is optionally complexed with a tertiary phosphine ligand, and
(e) a heterogeneous metal promoter used at a molar ratio of 0.05 to 100 moles of heterogeneous metal relative to the moles of soluble cobalt hydroformylation catalyst.

2. The process of claim 1 wherein the heterogeneous metal promoter is selected from the group consisting of Raney or sponge metal cobalt, Raney copper, Raney nickel, supported cobalt, supported copper, supported nickel, supported ruthenium, supported iron, and supported rhodium catalysts.

3. The process of claim 1 wherein the heterogeneous metal promoter is selected from the group consisting of Raney or sponge metal cobalt and supported cobalt, copper and ruthenium catalysts.

4. The process of claim 1 wherein the heterogeneous metal promoter is Raney or sponge metal cobalt.

5. The process of claim 4 wherein the heterogeneous metal promoter is Raney cobalt.

6. The process of claim 1 wherein the heterogeneous metal promoter is copper metal.

7. The process of claim 1 wherein the heterogeneous metal promoter is ruthenium metal.

8. The process of claim 1 wherein the hydroformylation reaction is carried out at a temperature of from 30 to 100° C. and a pressure of at least 0.8 MPa.

9. The process of claim 1 wherein the hydroformylation reaction is carried out at an initial temperature of from 30 to 100° C. and at a final temperature from 120 to 175° C.

10. The process of claim 1 wherein the oxirane is comprised of from 2 to 10 carbon atoms.

11. The process of claim 1 wherein the oxirane is ethylene oxide.

12. The process of claim 1 wherein the heterogeneous metal promoter is a solid.
